(19) United States
(12) Patent Application Publication
(10) Pub. No.: US 2011/0282100 A1
(43) Pub. Date: Nov. 17, 2011

(54) PROCESS FOR PREPARING MEMANTINE

(75) Inventors: Gunter Quack, Frankfurt am Main (DE); Markus-Rene Gold, Karlstadt (DE)

(73) Assignee: MERZ PHARMA GmbH & Co. KGaA, FRANKFURT am MAIN (DE)

(21) Appl. No.: 13/138,203

(22) PCT Filed: Jan. 20, 2010
(86) PCT No.: PCT/EP2010/000329

§ 371 Date: Jul. 19, 2011

Related U.S. Application Data
(60) Provisional application No. 61/205,481, filed on Jan. 21, 2009.

(30) Foreign Application Priority Data
Jan. 21, 2009 (EP) 090000795.6

(51) Int. Cl. C07C 209/62; C07C 13/615

(52) U.S. Cl. 564/459; 585/352

(57) ABSTRACT

The present invention relates to a process for preparing memantine, or a pharmaceutically acceptable salt thereof (e.g., memantine hydrochloride), which is substantially free of impurities.

### FIELD OF THE INVENTION

The present invention relates to a process for the synthesis of memantine, or a pharmaceutically acceptable salt thereof (e.g., memantine hydrochloride), which is substantially free of impurities.

### BACKGROUND OF THE INVENTION

Memantine (1-amino-3,5-dimethyl adamantane, disclosed, e.g., in U.S. Pat. Nos. 4,122,193; 4,273,774; 5,061,703) is a systemically-active uncompetitive NMDA receptor antagonist having moderate affinity for the receptor and strong voltage dependency and rapid blocking/unblocking kinetics. Memantine has been shown to be useful in alleviation of various progressive neurodegenerative disorders such as dementia in patients with moderate to severe Alzheimer's disease, Parkinson's disease, and spasticity (see, e.g., U.S. Pat. Nos. 5,061,703; 5,614,560, and 6,034,134; Parsons et al., Neuropharmacology 1999 June; 38(6):735-67; Möbius, ADAD, 1999, 13:S172-178; Danysz et al., Neurotox. Res., 2000, 2:85-97; Winblad and Poritis, Int. J. Geriatr. Psychiatry, 1999, 14:135-146; Danysz et al., Curr. Pharm. Des., 2002, 8:835-843; Jirgensons et. al., Eur. J. Med. Chem., 2000, 35: 555-565). Memantine has also been suggested to be useful in the treatment of AIDS dementia (U.S. Pat. No. 5,506,231), neuropathic pain (U.S. Pat. No. 5,334,618), epilepsy, glaucoma, hepatic encephalopathy, multiple sclerosis, stroke, tardive dyskinesia (Parsons et al., 1999, supra), autism, Attention-Deficit/Hyperactivity Disorder (ADHD) and other autistic spectrum disorders (US Published Application No. 2006/0079582). Memantine is currently approved in Europe and the United States for the treatment of Alzheimer's disease.

U.S. Pat. No. 3,391,142 discloses a process for the synthesis of adamantylamines, including memantine hydrochloride, involving treatment of 1-bromo-3,5-dimethyladamantane with acetonitrile and concentrated sulfuric acid to yield the corresponding 1-acetamido-3,5-dimethyladamantane which is hydrolyzed with sodium hydroxide to yield 1-amino-3,5-dimethyladamantane which is converted to memantine hydrochloride via treatment with hydrochloric acid.

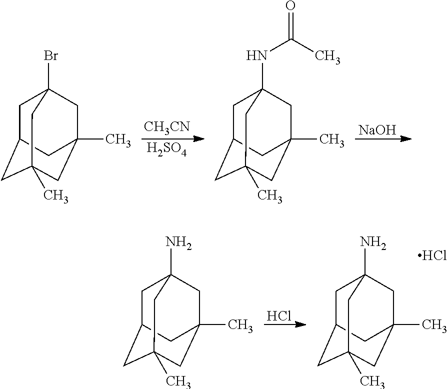

U.S. Pat. No. 4,122,193 discloses a process for the synthesis of 1-amino-3,5-dialkyl adamantane derivatives, including memantine hydrochloride, which involves treatment of a 1-halo-3,5-dialkyl adamantane derivatives with a urea followed by treatment with hydrochloric acid.

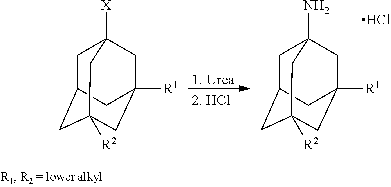

U.S. Pat. No. 5,061,703 discloses a process for the synthesis of aminoadamantanes, including memantine hydrochloride, which involves halogenation and/or alkylation of the adamantane ring followed by introduction of the amino group via treatment of the halogenated derivative with formamide and subsequent hydrolysis.

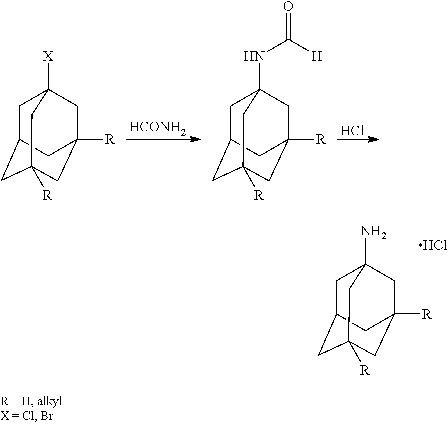

Czech Republic Patent No. 288445 discloses a process for the synthesis of 1-amino-3,5-dimethyladamantane hydrochloride wherein 1-chloro-3,5-dimethyladamantane is reacted with formamide followed by treatment of the formamide intermediate with aqueous hydrochloric acid to yield 1-amino-3,5-dimethyladamantane hydrochloride.

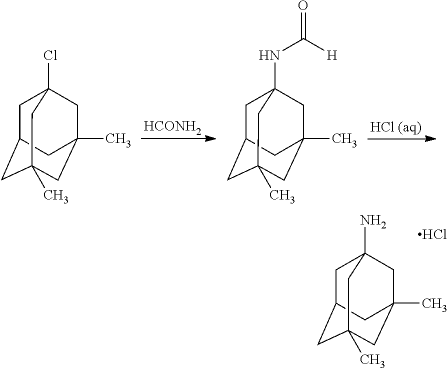

Czech Republic Patent No. 282398 discloses a process for the synthesis of 1-amino-3,5-dimethyladamantane hydrochloride which involves treatment of 1-acetamido-3,5-dimethyl adamantane with a base (such as postassium hydroxide) in a solvent such as methanol, ethanol, or 2-propanol.

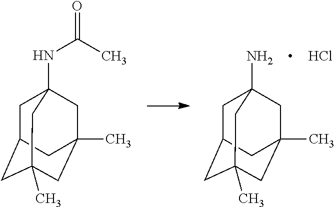

Chinese Published Patent Publication No. CN 1566075 discloses a process for preparing 1-aminoadamantane derivatives, including memantine hydrochloride, wherein a halogenated adamantane compound (which may have substituents, including alkyl groups, in the 3 and 5 positions) is reacted with formamide or a substituted formamide, followed by deformylation under acidic conditions to yield a 1-aminoadamantane derivative.

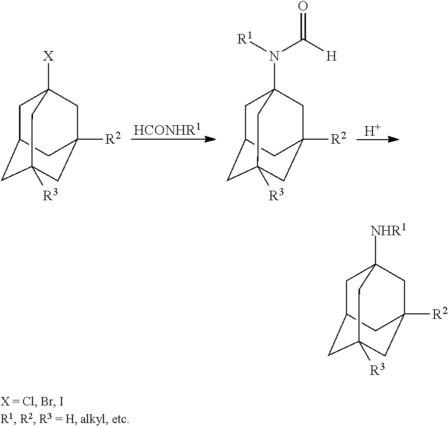

U.S. Pat. No. 5,599,998 discloses a process for the synthesis of 1-aminoadamantane derivatives, including memantine, which involves treatment of a 1-halo adamantane derivative with lithium metal to yield the lithiated intermediate which is treated with an aminating agent (such as NH₂Cl) under sonication conditions.

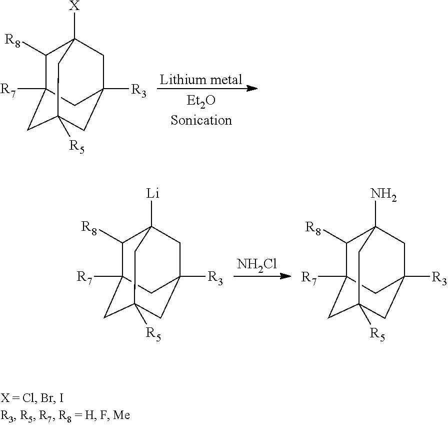

US Published Application No. 2006/025885 discloses a process for the synthesis of 1-aminoadamantane derivatives, including memantine hydrochloride, wherein a halogenated adamantane compound (which may have alkyl substituents, in the 3 and 5 positions of the adamantane ring) is reacted with acetonitrile in the presence of glacial acetic acid and concentrated sulfuric acid, followed by hydrolysis in the presence of an alkaline earth metal in a solvent such as 1-methoxy-2-propanol to yield the 1-aminoadamantane derivative which may then be converted to an acid addition salt via treatment with the appropriate acid (e.g., hydrochloric acid).

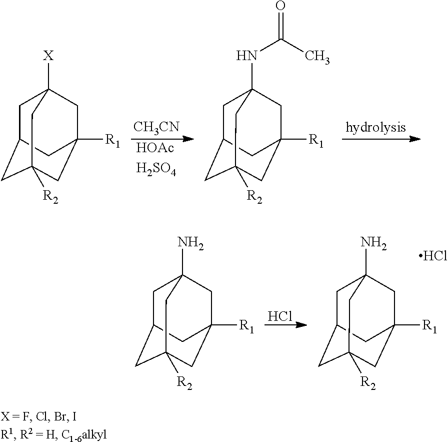

International Publication No. WO 2006/076562 discloses a process for the synthesis of memantine hydrochloride, wherein 1-halo-3,5-dimethyladamantane is reacted with acetonitrile in the presence of phosphoric acid to yield N-acetyl-1-amino-3,5-dimethyladamantane, which may be converted to memantine (for example, via basic hydrolysis) which may then be treated with hydrochloric acid to yield memantine hydrochloride.

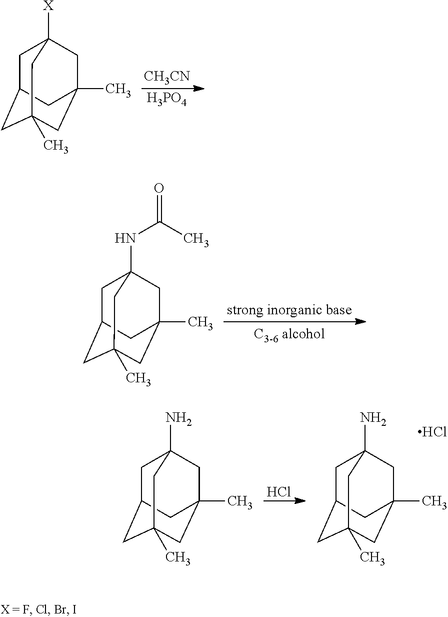

International Publication No. WO 2006/122238 discloses processes for preparing memantine or an acid addition salt of memantine, which involve either reaction of 1-bromo-3,5-dimethyladamantane with formamide to form N-formyl-1-amino-3,5-dimethyladamantane or reaction of 1-hydroxy-3,5-dimethyladamantane with a hydrogen halide to obtain 1-halo-3,5-dimethyl adamantane which is then reacted with formamide to yield N-formyl-1-amino-3,5-dimethyladamantane. The N-formyl-1-amino-3,5-dimethyladamantane intermediate is deformylated under acidic conditions to yield memantine hydrochloride.

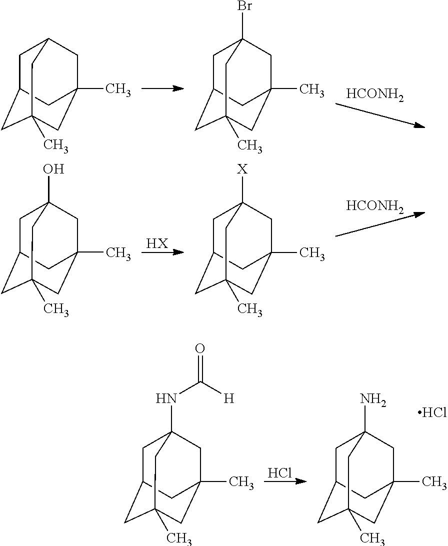

International Publication No. WO 2005/062724 discloses a process for the synthesis of 1-aminoadamantane derivatives, including memantine, which involves bromination of a compound of formula IIa, followed by hydrolysis to yield a compound of formula IId, which is treated with acetonitrile in the presence of an acid (e.g., sulfuric acid) to yield the acetamido intermediate of formula IIc. The compound of formula IIc is then hydrolyzed in the presence of acid or base to yield the aminoadamantane derivative of formula I.

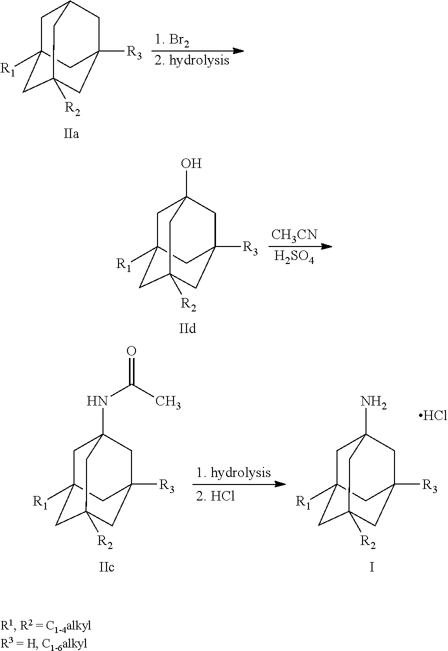

International Publication No. WO 2007/101536 discloses a process for the synthesis of 1-formamido-3,5-dimethyladmantane which involves treatment of 1,3-dimethyladamantane with formamide in a concentrated acid, as well as a process for the conversion of 1-formamido-3,5-dimethyladmantane to memantine hydrochloride via hydrolysis with hydrochloric acid.

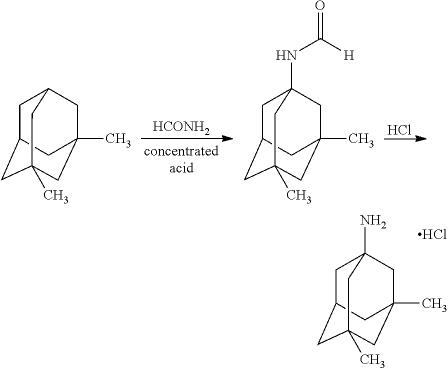

Pharmaceutical active ingredients must meet strict regulatory requirements with respect to purity. Typically, products meeting such purity requirements may obtained by purifying a crude active pharmaceutical ingredient using standard purification techniques.

Memantine produced by processes known in the art may contain trace impurities including 1-amino-3,5,7-trimethyladamantane. As such trace impurities are closely related to memantine, isolating memantine from crude preparations using standard purification techniques is difficult.

Thus, a need exists to develop a process for producing memantine which is substantially free of impurities, such as 1-amino-3,5,7-trimethyladamantane.

The instant inventors have discovered that by employing 1,3-dimethyladamantane which contains 0.05% or less of the impurity 1,3,5-trimethyladamantane in a conventional process for preparing memantine, or a pharmaceutically acceptable salt thereof, it is possible to obtain memantine which is substantially free of the impurity 1-amino-3,5,7-trimethyladamantane.

### SUMMARY OF THE INVENTION

The present invention relates to a process for the synthesis of memantine, or a pharmaceutically acceptable salt thereof, which is substantially free of the impurity 1-amino-3,5,7-trimethyladamantane, comprising reaction of 1,3-dimethyladamantane which contains 0.05% or less of the impurity 1,3,5-trimethyladamantane with an appropriate reagent or sequence of reagents to yield a 1-substituted-3,5-dimethyladamantane, wherein the substituent at the 1-position is a functional group which may be converted to an amino group (e.g., formamido, acetamido, or haloacetamido), which 1-substituted-3,5-dimethyladamantane is then converted to memantine or a pharmaceutically acceptable salt thereof.

A further aspect of the invention relates to a process for the synthesis of memantine, or a pharmaceutically acceptable salt thereof (e.g., memantine hydrochloride), which is substantially free of the impurity 1-amino-3,5,7-trimethyladamantane, comprising halogenation of 1,3-dimethyladamantane which contains 0.05% or less of the impurity 1,3,5-trimethyladamantane to yield 1-halo-3,5-dimethyladamantane, which compound is treated with formamide to yield 1-formamido-3,5-dimethyladamantane, wherein the 1-formamido-3,5-dimethyladamantane intermediate is subjected to basic hydrolysis to yield 1-amino-3,5-dimethyladamantane, which is substantially free of the impurity 1-amino-3,5,7-trimethyladamantane, which compound may be converted to a pharmaceutically acceptable salt via treatment with a pharmaceutically acceptable acid, or the 1-formamido-3,5-dimethyladamantane intermediate is subjected to acidic hydrolysis to yield 1-amino-3,5-dimethyladamantane, which is substantially free of the impurity 1-amino-3,5,7-trimethyladamantane, as an acid addition salt.

A further aspect of the invention relates to a process for the synthesis of memantine, or a pharmaceutically acceptable salt thereof (e.g., memantine hydrochloride), which is substantially free of the impurity 1-amino-3,5,7-trimethyladamantane, wherein 1,3-dimethyladamantane which contains 0.05% or less of the impurity 1,3,5-trimethyladamantane is treated with formamide in the presence of a concentrated acid to yield 1-formamido-3,5-dimethyladamantane, wherein the 1-formamido-3,5-dimethyladamantane intermediate is subjected to basic hydrolysis to yield 1-amino-3,5-dimethyladamantane, which is substantially free of the impurity 1-amino-3,5,7-trimethyladamantane, which compound may be converted to a pharmaceutically acceptable salt via treatment with a pharmaceutically acceptable acid, or the 1-formamido-3,5-dimethyladamantane intermediate is subjected to acidic hydrolysis to yield 1-amino-3,5-dimethyladamantane, which is substantially free of the impurity 1-amino-3,5,7-trimethyladamantane, as an acid addition salt.

A further aspect of the invention relates to a process for the synthesis of memantine, or a pharmaceutically acceptable salt thereof (e.g., memantine hydrochloride), which is substantially free of the impurity 1-amino-3,5,7-trimethyladamantane, comprising halogenation of 1,3-dimethyladamantane which contains 0.05% or less of the impurity 1,3,5-trimethyladamantane to yield 1-halo-3,5-dimethyladamantane, which compound is treated with acetonitrile in the presence of acid (e.g., sulfuric acid, phosphoric acid, nitric acid, a combination of acetic acid and sulfuric acid, or mixtures thereof) to yield 1-acetamido-3,5-dimethyladamantane, which compound is hydrolyzed to yield 1-amino-3,5-dimethyladamantane, which is substantially free of the impurity 1-amino-3,5,7-trimethyladamantane, which compound may be converted to a pharmaceutically acceptable salt via treatment with a pharmaceutically acceptable acid.

A further aspect of the invention relates to a process for the synthesis of memantine hydrochloride, which is substantially free of the impurity 1-amino-3,5,7-trimethyladamantane, comprising treatment of 1,3-dimethyladamantane which contains 0.05% or less of the impurity 1,3,5-trimethyladamantane with bromine to yield 1-bromo-3,5-dimethyladamantane, which compound is treated with formamide to yield 1-formamido-3,5-dimethyladamantane, wherein the 1-formamido-3,5-dimethyladamantane intermediate is hydrolyzed with hydrochloric acid to yield 1-amino-3,5-dimethyladamantane hydrochloride, which is substantially free of the impurity 1-amino-3,5,7-trimethyladamantane.

An additional aspect of the invention relates to such a process wherein the 1-amino-3,5-dimethyladamantane hydrochloride obtained is further purified by recrystallization from an appropriate solvent(s), such as water, C₁-C₄ alcohols (e.g., methanol, ethanol, isopropanol) and mixtures thereof.

A further aspect of the invention relates to a process for the synthesis of memantine, or a pharmaceutically acceptable salt thereof, wherein 1,3-dimethyladamantane which contains 0.05% or less of the impurity 1,3,5-trimethyladamantane is used as a starting material.

A further aspect of the invention relates to a process for the preparation of 1,3-dimethyladamantane which contains 0.05% or less of the impurity 1,3,5-trimethyladamantane comprising catalytic hydrogenation of acenaphthene at elevated temperature and pressure to yield perhydroacenaphthene, which compound is treated with a Lewis acid such as AlCl₃ and/or AlBr₃ in the presence or absence of HCl to yield 1,3-dimethyladamantane which is purified by fractional distillation.

A further aspect of the invention relates to the use of 1,3-dimethyladamantane which contains 0.05% or less of the impurity 1,3,5-trimethyladamantane in the synthesis of memantine or a pharmaceutically acceptable salt thereof, which is substantially free of the impurity 1-amino-3,5,7-trimethyladamantane.

An additional aspect of the invention relates to the use of 1,3-dimethyladamantane which contains 0.05% or less of the impurity 1,3,5-trimethyladamantane in the synthesis of memantine hydrochloride which is substantially free of the impurity 1-amino-3,5,7-trimethyladamantane.

### BRIEF DESCRIPTION OF THE DRAWINGS

FIG. 1 shows a gas chromatogram (GC) of a high purity sample of 1,3-dimethyladamantane.

### DETAILED DESCRIPTION OF THE INVENTION

A process for the preparation of 1,3-dimethyladamantane is shown in Scheme 1.

Acenaphthene (1) is hydrogenated over a catalyst such as Raney Nickel at elevated temperature and pressure to yield perhydroacenaphthene (2). Perhydroacenaphthene is treated with a Lewis acid such as AlCl₃ and/or AlBr₃ in the presence or absence of HCl to yield 1,3-dimethyladamantane which may be further purified (e.g., via fractional distillation) to provide 1,3-dimethyladamantane (4) containing 0.05% or less of the impurity 1,3,5-trimethyladamantane.

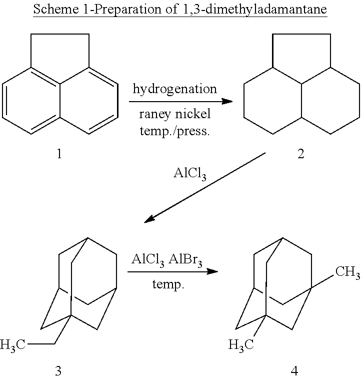

1,3-dimethyladamantane containing 0.05% or less of the impurity 1,3,5-trimethyladamantane may be converted to memantine, or a pharmaceutically acceptable salt thereof (e.g., memantine hydrochloride), which is substantially free of the impurity 1-amino-3,5,7-trimethyladamantane, according to Scheme 2.

1,3-dimethyladamantane (4), which contains 0.05% or less of the impurity 1,3,5-trimethyladamantane, may be treated with a halogenating agent (e.g., bromine, chlorine, or t-butylchloride) to yield 1-halo-3,5-dimethyladamantane derivative 5. Derivative 5 may be treated with formamide to yield 1-formamido-3,5-dimethyladamantane derivative 6. Alternatively, 1,3-dimethyladamantane, which contains 0.05% or less of the impurity 1,3,5-trimethyladamantane, may be treated with formamide in the presence of concentrated acid to yield 1-formamido-3,5-dimethyladamantane derivative 6. Derivative 6 may be hydrolyzed under basis or acidic conditions to provide memantine, which is substantially free of the impurity 1-amino-3,5,7-trimethyladamantane, or a pharmaceutically acceptable salt thereof.

Derivative 5 may be also treated with acetonitrile in the presence of acid (e.g., sulfuric acid, phosphoric acid, nitric acid, a combination of acetic acid and sulfuric acid, or mixtures thereof) to yield 1-acetamido-3,5-dimethyladamantane derivative 7. Derivative 7 may be hydrolyzed to provide memantine, which is substantially free of the impurity 1-amino-3,5,7-trimethyladamantane, which may be converted to a pharmaceutically acceptable salt via treatment with a pharmaceutically acceptable acid.

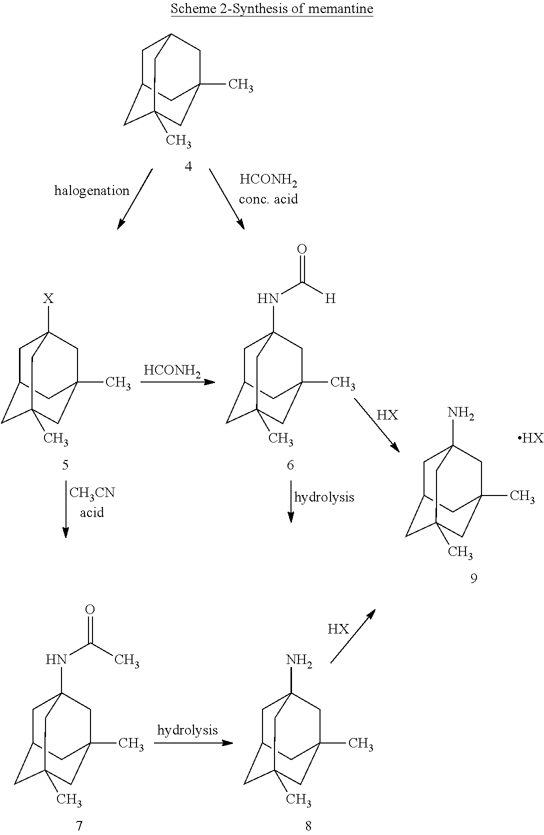

As used herein, the term halogen refers to fluorine, chlorine, bromine, and iodine.

As used herein, the term “substantially free of the impurity 1,3,5-trimethyladamantane” used in conjunction with 1,3-dimethyladamantane includes 1,3-dimethyladamantane which contains 0.05% or less of the impurity 1,3,5-trimethyladamantane.

As used herein, the term “substantially free of the impurity 1-amino-3,5,7-trimethyladamantane” used in conjunction with memantine (or a pharmaceutically acceptable salt thereof, e.g., memantine hydrochloride) includes memantine (or a pharmaceutically acceptable salt thereof, e.g., memantine hydrochloride) which contains 0.02% or less of the impurity 1-amino-3,5,7-trimethyladamantane.

As used herein, pharmaceutically acceptable salts include, but are not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity.

Memantine or a pharmaceutically acceptable salt thereof (e.g., memantine hydrochloride), which is substantially free of the impurity 1-amino-3,5,7-trimethyladamantane, prepared according to a process of the present invention may be formulated as a pharmaceutical composition.

Such pharmaceutical compositions may be in the form of a solid, semisolid, thin film/flash dose, or liquid formulation according to the following.

The compositions may be administered orally, topically, parenterally, or mucosally (e.g., buccally, by inhalation, or rectally) in dosage unit formulations containing conventional non-toxic pharmaceutically acceptable carriers. The compositions may be administered orally in the form of a capsule, a tablet, or the like, or as a semi-solid, thin film/flash dose, or liquid formulation (see Remington's Pharmaceutical Sciences, 20^{th} Edition, by A. R. Gennaro).

For oral administration in the form of a tablet or capsule, the compositions may be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets may be coated with a concentrated sugar solution which may contain e.g., gum arabic, gelatine, talcum, titanium dioxide, and the like. Alternatively, the tablets can be coated with a polymer that dissolves in a readily volatile organic solvent or mixture of organic solvents. In specific embodiments, the active substances are formulated in immediate-release (IR) or modified-release (MR) tablets. Immediate release solid dosage forms permit the release of most or all of the active ingredient over a short period of time, such as 60 minutes or less, and make rapid absorption of the drug possible (for example, immediate release formulations of memantine are disclosed in US Published Application Nos. 2006/0002999 and 2007/0065512, the subject matter of which is hereby incorporated by reference). Modified release solid oral dosage forms permit the sustained release of the active ingredient over an extended period of time in an effort to maintain therapeutically effective plasma levels over similarly extended time intervals and/or to modify other pharmacokinetic properties of the active ingredient (for example, modified release formulations of memantine are disclosed in US Published Application Nos. 2006/0051416 and 2007/0065512, the subject matter of which is hereby incorporated by reference).

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The compositions of the invention can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Formulation of the compositions of the invention in a semi-solid or liquid form may also be used. The active ingredient (i.e., memantine or a pharmaceutically acceptable salt thereof) may constitute between 0.1 and 99% by weight of the formulation, more specifically between 0.5 and 20% by weight for formulations intended for injection and between 0.2 and 50% by weight for formulations suitable for oral administration.

In one embodiment of the invention, the compositions are administered in modified release formulations. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks.

A modified release form dosage may comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, the compositions are formulated in oral, liquid formulations. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. For example, oral liquid formulations of memantine are described in PCT Application No. PCT/US2004/037026, the subject matter of which is hereby incorporated by reference.

For oral administration in liquid form, the compositions may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of the active substance, with the balance being sugar and mixture of ethanol, water, glycerol and propylene glycol. Optionally, such liquid formulations may contain coloring agents, flavoring agents, saccharine and carboxymethyl-cellulose as a thickening agent or other excipients.

In another embodiment, a therapeutically effective amount of the active substance is administered in an oral solution containing a preservative, a sweetener, a solubilizer, and a solvent. The oral solution may include one or more buffers, flavorings, or additional excipients. In a further embodiment, a peppermint or other flavoring is added to the oral liquid formulation.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection may be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

The formulations of the invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient may be in powder form for reconstitution with a suitable vehicle, e.g., sterile pyrogen-free water, before use.

The invention also provides a pharmaceutical pack or kit comprising one or more containers containing the active substances (i.e., memantine or a pharmaceutically acceptable salt thereof) and, optionally, more of the ingredients of the formulation. In a specific embodiment, the compositions are provided as oral solutions (2 mg/ml) for administration with the use of a 2 teaspoon capacity syringe (dosage KORC®). Each oral syringe has blue hatch marks for measurement, with lines on the right side of the syringe (tip down) representing tsp units, and those on the left representing ml units.

The optimal therapeutically effective amount may be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Dosage units for rectal application may be solutions or suspensions or may be prepared in the form of suppositories or retention enemas comprising the active substances in a mixture with a neutral fatty base, or gelatin rectal capsules comprising the active substances in admixture with vegetable oil or paraffin oil.

### EXAMPLES

The following examples illustrate the invention without limiting its scope.

### Example 1

### Analytical Method for Determining Impurities in 1,3-dimethyladamantane

Analysis is performed on DANI 86.10 HT gas chromatograph equipped with CTC 200S automatic sampler. Millennium 3.20 software is applied for acquisition and processing of the data. All further calculations are made by MS Excel 97 software, statistics are calculated according to “Validierung in Chromotographie” Novia GmbH. 8 Feb. 1996, Frankfurt am Main.

Gas chromatographic analysis to determine the impurity profile of 1,3-dimethyladamantane is performed on an SE-52 capillary column (length: 25; ID: 0.32 mm; d_{f}=0.25 μm). An flame ionization detector (FID) is applied for detection. The injector temperature is 250° C. and the detector temperature is also 250° C. The carrier gas is nitrogen.

Both calibration standards and analytical samples are prepared by dissolution of the components in hexane. 1-Hydroxyadamantane is applied as internal standard. All impurities are calibrated against the 1,3-dimethyladamantane peak.

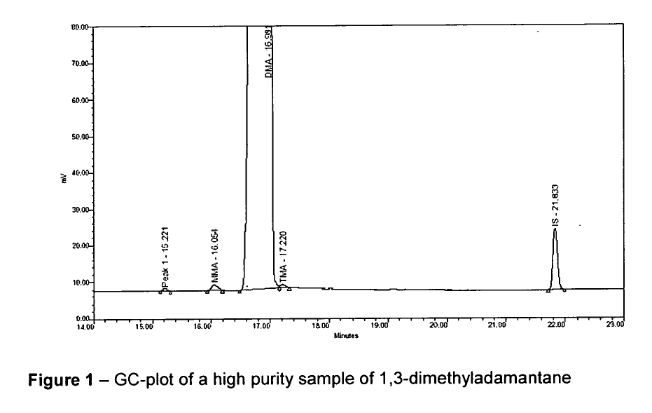

Results for a high purity sample of 1,3-dimethyladamantane are shown in Table 1 below and in FIG. 1.

TABLE 1
Impurity profile for high purity 1,3-dimethyladamantane
Impurity content, % of DMA (calcd. as DMA)

| Unidentified peak | | | | | | Unidentified | Unidentified |
|---|---|---|---|---|---|---|---|
| RT = 15.2 min | MMA | TMA | Z-1,4-DMA | E-1,4-DMA | ETA | peak RT = 18.8 | peak RT = 20.2 |
| 0.013 | 0.049 | 0.024 | — | — | — | — | — |

### Example 2

### Preparation of 1,3-dimethyladamantane

Acenaphthene is washed with bentonite in toluene, followed by a second wash with cyclohexene. The acenaphthene is then hydrogenated over Raney Nickel at 150 bar at a temperature of 140 to 180° C. The catalyst is filtered off and the crude perhydroacenaphthene (240 kg) is treated with AlCl₃ (45 kg) and HCl (100 mL) at 80 to 90° C. for 4 h. The reaction mixture is then heated to 120° C. for 8 h. An additional 100 mL of hydrochloric acid and an additional 5 kg of AlCl₃ are added and the reaction mixture is heated to 80 to 90° C. for 4 h. The crude 1,3-dimethyladamantane is purified via fractional distillation on a DN 300 column with oriented Sulzer type packing providing a minimum of 60 theoretical plates, with the temperature profile being dependent on column pressure. The critical point is estimated on the basis of trend and is confirmed by analytical control. 1,3-Dimethyladamantane containing 0.05% or less of the impurity 1,3,5, trimethyladamantane is obtained in about 75% yield.

### Example 3

### Preparation of 1-bromo-3,5-dimethyladamantane

1,3-dimethyladamantane containing 0.05% or less of the impurity 1,3,5, trimethyladamantane, as prepared in Example 2, is treated with bromine (3 equivalents) and heated to reflux for 16 h. The reaction mixture is cooled to about 15° C. and quenched with sodium bisulphite in methylene chloride. The aqueous layer is removed, and the organic layer is washed with water. The organic layer is concentrated in vacuo to yield 1-bromo-3,5-dimethyladamantane as an oil.

### Example 4

### Preparation of 1-amino-3,5-dimethyladamantane hydrochloride

1-bromo-3,5-dimethyladamantane as prepared in Example 3, is treated with an excess of formamide and heated to 120° C. for 3 to 5 h. The reaction mixture is cooled and diluted with methylene chloride. This mixture is washed 4 times with a 30% sodium hydroxide solution. The organic layer is concentrated via distillation followed by addition of water. The distillation is continued to remove the organic layer, and the mixture is then cooled to below 80° C. The N-formyl-1-amino-3,5-dimethyladamantane intermediate, which is optionally isolated, is then hydrolyzed by addition of a 37% hydrochloric acid solution. The reaction mixture is heated to reflux for about 3 h, and the reaction mixture is then cooled to 5° C. to yield crude 1-amino-3,5-dimethyladamantane hydrochloride which is isolated by centrifugation and washed with water followed by ethyl acetate. The crude 1-amino-3,5-dimethyladamantane hydrochloride is then reprecipitated to yield the title compound.

The purity of memantine hydrochloride prepared according to Examples 3-4 is shown in Table 2.

TABLE 2

| 1,3-dimethyladamantane | Memantine |
|---|---|
| 1,3,5-trimethyladamantane | 1-amino-3,5,7-trimethyladamantane |
| (TMA) content in % | (TMM) content in % |
| 0.03 | 0.01 |

### Example 5

### Preparation of 1-formamido-3,5-dimethyladamantane

1,3-dimethyladamantane containing 0.05% or less of the impurity 1,3,5, trimethyladamantane, as prepared in Example 2, is treated with nitric acid followed by sulfuric acid at 0° C. The reaction is stirred over night at 0° C. The reaction mixture is poured onto 100 mL formamide (at 0° C.) in a round bottom flask which is equipped with a drying tube. The reaction is stirred at 0° C. for 30 min and then at room temperature for 90 min. Dichloromethane and water are then added. The organic phase is removed and washed with water and a 2% NaHCO₃-solution, dried over Na₂SO₄ and concentrated in vacuo. The resulting oil is purified via column chromatography to yield the title compound as a solid.

### Example 6

### Preparation of 1-amino-3,5-dimethyladamantane

1-formamido-3,5-dimethyladamantane, as prepared in Example 5, is hydrolyzed by addition of a 37% hydrochloric acid solution. The reaction mixture is heated to reflux for about 3 h, and the reaction mixture is then cooled to 5° C. to yield crude 1-amino-3,5-dimethyladamantane hydrochloride which is filtered and washed with water followed by ethyl acetate. The crude 1-amino-3,5-dimethyladamantane hydrochloride is then reprecipitated to yield the title compound.

### Example 7

### Relationship Between Content of the 1-amino-3,5,7-trimethyladamantane (TMM) Impurity in Memantine and the 1,3,5-trimethyladamantane impurity in 1,3-dimethyladamantane

Memantine is synthesized according to Examples 5-6 starting from 1,3-Dimethyladamantane (1,3-DMA) spiked with different levels of the alkyl adamantane impurity 1,3,5-trimethyladamantane (TMA). The level of the corresponding aminoalkyl adamantane impurity 1-amino-3,5,7-trimethyladamantane (TMM) in the final memantine product is determined. The results (shown in Table 3) demonstrate that the amount of the 1-amino-3,5,7-trimethyladamantane (TMM) impurity in the memantine final product is dependent on the amount of 1,3,5-trimethyladamantane (TMA) impurity present in the 1,3-dimethyladamantane starting material.

TABLE 3

| 1,3-dimethyladamantane | Memantine |
|---|---|
| 1,3,5-trimethyladamantane | 1-amino 1,3,5-trimethyladamantane |
| (TMA) content in % | (TMM) content in % |
| 0.25 | 0.07 |
| 0.42 | 0.14 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1. A process for the synthesis of memantine, or a pharmaceutically acceptable salt thereof, which is substantially free of the impurity 1-amino-3,5,7-trimethyladamantane, comprising reaction of 1,3-dimethyladamantane which contains 0.05% or less of the impurity 1,3,5-trimethyladamantane with an appropriate reagent or sequence of reagents to yield a 1-substituted-3,5-dimethyladamantane, wherein the substituent at the 1-position is a functional group which may be converted to an amino group, which 1-substituted-3,5-dimethyladamantane is then converted to memantine or a pharmaceutically acceptable salt thereof.

2. The process of claim 1, wherein the functional group which may be converted to an amino group is selected from formamido, acetamido, and haloacetamido.

3. The process of claim 1, wherein 1,3-dimethyladamantane which contains 0.05% or less of the impurity 1,3,5-trimethyladamantane is halogenated to yield 1-halo-3,5-dimethyladamantane, wherein the 1-halo-3,5-dimethyladamantane intermediate is treated with formamide to yield 1-formamido-3,5-dimethyladamantane, wherein the 1-formamido-3,5-dimethyladamantane intermediate is subjected to hydrolysis to yield 1-amino-3,5-dimethyladamantane, which compound may be converted to a pharmaceutically acceptable salt via treatment with a pharmaceutically acceptable acid.

4. The process of claim 1, wherein 1,3-dimethyladamantane which contains 0.05% or less of the impurity 1,3,5-trimethyladamantane is treated with formamide in the presence of a concentrated acid to yield 1-formamido-3,5-dimethyladamantane, wherein the 1-formamido-3,5-dimethyladamantane intermediate is subjected to hydrolysis to yield 1-amino-3,5-dimethyladamantane, which compound may be converted to a pharmaceutically acceptable salt via treatment with a pharmaceutically acceptable acid.

5. The process of claim 1, wherein 1,3-dimethyladamantane which contains 0.05% or less of the impurity 1,3,5-trimethyladamantane is halogenated to yield 1-halo-3,5-dimethyladamantane, which compound is treated with acetonitrile in the presence of acid to yield 1-acetamido-3,5-dimethyladamantane, which compound is hydrolyzed to yield 1-amino-3,5-dimethyladamantane, which compound may be converted to a pharmaceutically acceptable salt via treatment with a pharmaceutically acceptable acid.

6. A process for the synthesis of memantine hydrochloride, comprising bromination of 1,3-dimethyladamantane which contains 0.05% or less of the impurity 1,3,5-trimethyladamantane to yield 1-bromo-3,5-dimethyladamantane, which compound is treated with formamide to yield 1-formamido-3,5-dimethyladamantane, wherein the 1-formamido-3,5-dimethyladamantane intermediate is hydrolyzed with hydrochloric acid to yield 1-amino-3,5-dimethyladamantane hydrochloride.

7. The process of claim 6, wherein 1-amino-3,5-dimethyladamantane hydrochloride is further purified by recrystallization from an appropriate solvent(s).

8. The process of claim 7, wherein the solvent is selected from methanol, ethanol, and mixtures thereof.

9. A process for the synthesis of memantine, or a pharmaceutically acceptable salt thereof, wherein 1,3-dimethyladamantane which contains 0.05% or less of the impurity 1,3,5-trimethyladamantane is used as a starting material.

10. A process for the preparation of 1,3-dimethyladamantane which contains 0.05% or less of the impurity 1,3,5-trimethyladamantane comprising catalytic hydrogenation of acenaphthene at elevated temperature and pressure to yield perhydroacenaphthene, which compound is treated with a Lewis acid to yield 1,3-dimethyladamantane which is purified by fractional distillation to yield 1,3-dimethyladamantane which contains 0.05% or less of the impurity 1,3,5-trimethyladamantane.

11. (canceled)

12. (canceled)
